(19) United States
(12) Patent Application Publication
(10) Pub. No.: US 2025/0312356 A1
(43) Pub. Date: Oct. 9, 2025

(54) TREATMENT OF DYSMENORRHEA

(71) Applicant: VISTAGEN THERAPEUTICS, INC., South San Francisco, CA (US)

(72) Inventor: Louis MONTI, South San Francisco, CA (US)

(21) Appl. No.: 19/172,408

(22) Filed: Apr. 7, 2025

Related U.S. Application Data
(60) Provisional application No. 63/631,434, filed on Apr. 8, 2024.
(60) Provisional application No. 63/631,389, filed on Apr. 8, 2024.
(60) Provisional application No. 63/711,496, filed on Oct. 24, 2024.

(51) Int. Cl. A61K 31/58; A61K 9/00; A61K 31/565; A61K 31/568; A61K 31/57; A61P 29/00

(52) U.S. Cl. CPC A61K 31/58; A61K 9/0043; A61K 31/565; A61K 31/568; A61K 31/57; A61P 29/00

(57) ABSTRACT

Methods and compositions for treating dysmenorrhea are disclosed. Included are methods for the intranasal administration of 16α,17α-epoxyestr-4-en-10β-ol-3-one, and compositions containing 16α,17α-epoxyestr-4-en-10β-ol-3-one that are formulated for intranasal delivery.

### CROSS-REFERENCE TO RELATED APPLICATIONS

This application claims priority to U.S. Application No. 63/631,434, filed Apr. 8, 2024; U.S. Application No. 63/631,389, filed Apr. 8, 2024; and U.S. Application No. 63/711,496, filed Oct. 24, 2024, the disclosures of which are incorporated herein by reference.

### BACKGROUND OF THE INVENTION

Dysmenorrhea is defined as pain during the menstrual cycle, usually located in the lower abdomen and sometimes radiating to the inner thighs and back. It is characterized by pelvic pain associated with menstruation and other symptoms that may present with severity. Nagy, et al., “Dysmenorrhea,” StatPearls—NCBI Bookshelf (2023).

Dysmenorrhea leads to decreased quality of life, absenteeism, and increased risk of depression and anxiety. The ACOG Committee Opinion Number 760, “Dysmenorrhea and Endometriosis in the Adolescent”, Obstet, Gynecol., 132(6), e249-e258 (2018), says that “[D]ysmenorrhea, or menstrual pain, is the most common menstrual symptom among adolescent girls and young women. Prevalence rates vary but range from 50% to 90%.”

Dysmenorrhea is also associated with psychological distress. Matsumura et al., “The association between the severity of dysmenorrhea and psychological distress of women working in central Tokyo—a preliminary study,” Int. J. Environ. Res. Public Health 20:7021 (2023); Rogers et al., “Dysmenorrhea and psychological stress: a meta-analysis,” Arch Womens Ment Health. 2023 December;26 (6): 719-735.

Nearly one-half of patients (45%) with symptoms of dysmenorrhea will present first to their primary care physician. Up to one-half of patients with dysmenorrhea miss school or work at least once, and 10% to 15% have regular absences during menses. A prospective longitudinal study of 400 patients with dysmenorrhea revealed that most have persistent symptoms throughout their years of menstruation, although some improvement in severity may occur, for example, after childbirth.“

Because of its high prevalence, the extent of its daily interference on functioning during menstruation, and its long-term effects on pain sensitivity, dysmenorrhea is a condition of significant concern for a high proportion of women.

### The Pharmacological Treatment of Dysmenorrhea

Mardon et al., “Investigational drugs for the treatment of dysmenorrhea”, Expert Opin. Invest. Drugs, DOI: 10.1080/13543784.2024.2326627, note (citations omitted) that “[C]urrent medical therapies for dysmenorrhea are dependent on the underlying cause of dysmenorrhea, if known. However as primary dysmenorrhea is a diagnosis of exclusion, treatment response is often part of the diagnostic process. First line treatments for dysmenorrhea, irrespective of the cause, include nonsteroidal anti-inflammatory drugs (NSAIDs) and the oral contraceptive pill, in particular for those wanting contraceptive effects.”

NSAIDs generally used for the treatment of dysmenorrhea are cyclooxygenase inhibitors, which thus inhibit prostaglandin synthetase and the formation of prostaglandins. Mardon et al. comment (citations omitted) that “[I]buprofen may be the optimal NSAID for primary dysmenorrhea” and that “[P]aracetamol, a common over the counter medication, does not appear to be any more effective than a placebo against primary dysmenorrhea and significantly less effective than NSAIDs.” They comment that gastritis and renal impairments can occur with long-time use of NSAIDs and that overuse of paracetamol (acetaminophen) is associated with liver toxicity and impaired kidney function.

Mardon et al. also note that hormonal therapies, including the combined (progestin/estrogen) contraceptive pill (COCP) and progestin-only options are also recommended as first-line treatments, and that “[T]here is high-quality evidence demonstrating the efficacy of COCPs for primary dysmenorrhea.” But they comment that the use of hormonal therapies has some limitations, noting that common side effects include headache, nausea, breast tenderness, genital irritation, tiredness, and bloating, and that there is an increased risk of venous thromboembolism, a very small increased risk of myocardial infarction and ischemic stroke, and a small increased risk of breast cancer and cervical cancer.

Second-line treatments for secondary dysmenorrhea associated with endometriosis include gonadotropin-releasing hormone (GnRH) analogs; but these are associated with menopausal symptoms and have negative effects on bone density.

By contrast, the first-line pharmacological treatment for premenstrual dysphoric disorder (PMDD), which is associated directly with poor mood, is selective serotonin reuptake inhibitors (SSRIs); while other treatments include oral contraceptives, and GnRH analogs, and diuretics. Non-pharmacological treatments include cognitive behavioral therapy.

Notwithstanding the present therapeutic agents, there still exists a need for effective and tolerable therapy for dysmenorrhea.

### Nasal Chemosensory Neurons and Pherines

In most species, including humans, neural chemosensory neurons cells (NCNs) in the olfactory mucosal lining of the dorsal nasal recess and lateral wall of the nasal cavity (opposite the septum), including the area or region of the vomeronasal organ (“VNO”, a chemosensory organ found in most vertebrates including humans) have chemosensory receptors associated with olfactory external chemosignals detection. See, generally, Lubke et al., “Always follow your nose: the functional significance of social chemosignals in human reproduction and survival,” Hormones and Behavior 68:134-144 (2015); Monti-Bloch et al., “Effect of putative pheromones on the electrical activity of the human vomeronasal organ and olfactory epithelium”, J. Steroid Biochem. Mol. Biol., vol. 39(4), pp. 573-582 (1991); Monti-Bloch et al., “The Human Vomeronasal System: A Review”, Ann. N.Y. Acad. Sci., vol. 855, pp. 373-389 (1998). The axons of the neuroepithelial cells of the nasal chemosensory receptors, including the VNO, have oligosynaptic input to the hypothalamus and limbic amygdala of the brain, while the distal processes (microvilli) have chemosensory receptors (Stensaas et al., “Ultrastructure of the human vomeronasal organ”, J. Steroid Biochem. Mol. Biol., vol. 39(4), pp. 553-560 (1991).

Human external chemosignals delivered to the nasal septal area bind to local chemosensory receptors and trigger nerve signals that reach the brain inducing physiological and behavioral changes (Grosser et al., “Behavioral and electrophysiological effects of androstadienone, a human pheromone”, Psychoneuroendocrinology, vol. 25, pp. 289-299 (2000).

Synthetic analogs of human external chemosignals called pherines (odorless substances that bind to nasal chemosensory receptors, including receptors in the VNO) can induce robust physiological, pharmacological, and behavioral effects when delivered airborne to these receptors via the nasal passages. This information is supported by several studies in human volunteers using functional magnetic resonance imaging and positron emission tomography, showing that pherines selectively activate the brain areas (hypothalamus, limbic system, cingulate gyrus, anterior thalamus and prefrontal cortex) where their physiological, pharmacological and behavioral effects are integrated.

Studies with several pherines have shown that, because the compounds act directly as agonists NCNs that are connected to the brain, via the olfactory bulbs, by short neural circuits, administration of the compounds causes an effect on physiological markers (for example, autonomic nervous system responses and EEG) within seconds to less than a minute, and an effect on behavior and endocrine and neurotransmitter metabolite markers within about 10-15 minutes.

### SUMMARY OF THE INVENTION

This invention treats dysmenorrhea by intranasal administration of 16α,17α-epoxyestr-4-en-10β-ol-3-one.

In other aspects, this invention thus includes:
16α, 17α-epoxyestr-4-en-10β-ol-3-one for the treatment of dysmenorrhea by intranasal administration; pharmaceutical formulations and devices containing 16α, 17α-epoxyestr-4-en-10β-ol-3-one for the treatment of dysmenorrhea by intranasal administration; and
the use of 16α,17α-epoxyestr-4-en-10β-ol-3-one in the manufacture of medicaments for the treatment of dysmenorrhea by intranasal administration.

16α,17α-Epoxyestr-4-en-10β-ol-3-one has particular utility in the treatment of dysmenorrhea (treatment of individuals suffering from dysmenorrhea); and is expected to have the following advantages over conventional treatments:
(1) ease of administration while retaining the ability for acute use;
(2) rapid onset of effect, because of the direct local delivery of the compound to nasal chemosensory receptors and consequent action. Current oral agents used in the treatment of dysmenorrhea, like other oral agents in general, may take 30 minutes or more for therapeutic effectiveness to be achieved; and
(3) lack of local nasal adverse effects and lack of systemic effects or toxicity, because of the local route of administration, the effect on peripheral (nasal) chemosensory receptors and the demonstrated lack of systemic bioavailability, and the effect on the central nervous system through the activation of short (oligosynaptic) neural circuits.

16α,17α-epoxyestr-4-en-10β-ol-3-one may be administered to the chemosensory neurons of the olfactory epithelium.

In one embodiment, the dysmenorrhea is primary or secondary dysmenorrhea.

In another embodiment, 16α,17α-epoxyestr-4-en-10β-ol-3-one is administered at the onset of a symptom of dysmenorrhea, or 1, 2, 3, 4, or 5 days before the expected start of a symptom or symptoms of dysmenorrhea.

16α,17α-epoxyestr-4-en-10β-ol-3-one may be administered from 1 to 6 times per day, for example, 2 to 6 times per day, 3 to 5 times per day, or 4 times per day.

The administration of 16α,17α-epoxyestr-4-en-10β-ol-3-one may be chosen to maximize the administration at the time when the expected occurrence of a symptom or symptoms of dysmenorrhea is most frequent or most severe in the dysmenorrhea symptomatic population.

The administration of 16α,17α-epoxyestr-4-en-10β-ol-3-one may be chosen to maximize the administration at the time when the expected occurrence of a symptom or symptoms of dysmenorrhea is most frequent or most severe in the individual being treated.

16α,17α-Epoxyestr-4-en-10β-ol-3-one may be administered in a nasal spray.

16α,17α-epoxyestr-4-en-10β-ol-3-one may be administered in a nasal spray comprising an aqueous solution of 16α,17α-epoxyestr-4-en-10β-ol-3-one .

16α,17α-epoxyestr-4-en-10β-ol-3-one may be administered in a nasal spray in a dosage of 0.8 to 6.4 μg 16α,17α-epoxyestr-4-en-10β-ol-3-one per administration, for example, 1.6 to 3.2 μg 16α,17α-epoxyestr-4-en-10β-ol-3-one per administration or 1.6 μg 16α,17α-epoxyestr-4-en-10β-ol-3-one per administration.

In another aspect, the invention provides methods for treating the symptoms of anxiety, depression, or psychological distress that are associated with dysmenorrhea in some subjects by administering a therapeutic amount of fasedienol or itruvone either alone or co-administered with a therapeutic amount of 16α,17α-epoxyestr-4-en-10β-ol-3-one.

In another aspect, the invention provides a method for improving the cognitive performance or psychomotor performance in subjects who have mental fatigue associated with dysmenorrhea, by the administration of a therapeutic amount PH15 (1,3,5(10),16-estratetraen-3-yl acetate) either alone or co-administered with a therapeutic amount of 16α,17α-epoxyestr-4-en-10β-ol-3-one .

In yet another aspect, the invention provides a nasal spray device for the administration of 16α,17α-epoxyestr-4-en-10β-ol-3-one packaged together with a label for the symptomatic or prophylactic treatment of the symptoms of dysmenorrhea.

### BRIEF DESCRIPTION OF THE DRAWINGS

FIG. 1 depicts improvement of cramping symptoms in a premenstrual dysphoric disorder (PMDD) clinical study; PH80=16α,17α-epoxyestr-4-en-10β-ol-3-one.

FIG. 2 depicts improvement of headache symptoms in a premenstrual dysphoric disorder (PMDD) clinical study; PH80=16α,17α-epoxyestr-4-en-10β-ol-3-one.

FIG. 3 depicts improvement of cramps in pain symptom cluster analysis from a daily symptom report in a Phase 2a study; PH80=16α,17α-epoxyestr-4-en-10β-ol-3-one .

### DETAILED DESCRIPTION OF THE INVENTION

The invention is described below, with reference to detailed illustrative embodiments. It will be apparent that the invention may be embodied in a wide variety of forms, some of which may be quite different from those of the disclosed embodiments. Consequently, the specific details disclosed below are merely representative and do not limit the scope of the invention.

The present invention is based on the inventors' clinical findings that certain doses of the steroidal pherine drug, 16α,17α-epoxyestr-4-en-10β-ol-3-one , effectively treat one or more of the symptoms of dysmenorrhea. Thus, the present invention relates to 16α, 17α-epoxyestr-4-en-10β-ol-3-one compositions and therapeutic methods involving administering such compositions to treat dysmenorrhea.

### Dysmenorrhea

“Dysmenorrhea” is described as “a syndrome of painful menses,” according to The American Psychiatric Association's Diagnostic and Statistical Manual of Mental Disorders, Fifth Edition (“DSM.5”). The DSM.5 notes that dysmenorrhea “is distinct from a syndrome characterized by affective changes,” such as PMDD which is a depressive (affective) psychological disorder that is discussed in more detail below.

Dysmenorrhea is generally associated with radiating abdominal pain around the time of menses. Pain may occur with menses or precede menses by 1 to 3 days. The pain tends to peak 24 hours after onset of menses and subside after 2 to 3 days. It is often crampy or a dull constant ache but may be sharp or throbbing and radiate to the back or legs. Headache, nausea, constipation or diarrhea, lower back pain, and urinary frequency are common symptoms of dysmenorrhea, and vomiting occurs occasionally. It is often accompanied by disruption of daily functional activities, including family care, social interactions, and occupational activity.

In some women, dysmenorrhea is accompanied by certain symptoms similar to those of premenstrual syndrome, premenstrual dysphoric disorder, or heavy menstrual bleeding and passage of blood clots. In about 5 to 15% of women with primary dysmenorrhea, cramps are severe enough to interfere with daily activities and may result in absence from school or work. Pain sensitivity with dysmenorrhea may increase susceptibility to other chronic pain conditions in later life. Mckenna and Fogleman, Am. Fam. Physician, 104(2), 164-170 (2021), report (citations omitted) “Dysmenorrhea, which is defined as painful menstruation, affects up to 50% to 90% of adolescent girls and women of reproductive age.

Dysmenorrhea may be categorized as primary or secondary. According to McKenna and Fogleman: “Primary dysmenorrhea occurs in the absence of pelvic pathology. It is mediated by elevated prostaglandin and leukotriene levels, with inflammation causing uterine contractility and cramping pain. Secondary dysmenorrhea is due to pelvic pathology or a recognized medical condition and accounts for about 10% of cases of dysmenorrhea. The most common etiology is endometriosis. Other etiologies include congenital or acquired obstructive and nonobstructive anatomic abnormalities (for example, Müllerian malformations, uterine leiomyomas, adenomyosis), pelvic masses, and infection.”

Mckenna and Fogleman also note that “Dysmenorrhea is typically described as cramping pain in the lower abdomen beginning at the onset of menstrual flow and lasting eight to 72 hours. It is often accompanied by nausea, vomiting, diarrhea, headaches, muscle cramps, low back pain, fatigue, and, in more severe cases, sleep disturbance. In a study of more than 400 patients with dysmenorrhea, 47% reported moderate pain, and 17% reported severe pain on a 0 to 10 visual analog scale. Primary dysmenorrhea begins an average of six to 12 months following menarche, corresponding with the initiation of ovulatory cycles, and tends to recur with every menstrual cycle. Symptoms of secondary dysmenorrhea may start immediately following menarche or may arise later in life. Symptoms more consistent with secondary dysmenorrhea include changes in or progressive worsening of pelvic pain, abnormal uterine bleeding, vaginal discharge, and dyspareunia.”

Primary dysmenorrhea is defined as painful, spasmodic cramping in the lower abdomen, just before and/or during menstruation, in the absence of any discernable macroscopic pelvic pathology. lacovides et al. The onset of primary dysmenorrhea usually occurs in adolescence, at or shortly after (6-24 months) menarche. The onset of primary dysmenorrheic pain usually has a clear and predictable temporal pattern, beginning just before or at the start of menstruation. The pain typically lasts for 8-72 h, is most severe during the first or second day of menstruation and may radiate to the back and thighs.

In addition, systemic symptoms such as nausea, vomiting, diarrhea, fatigue and insomnia frequently accompany the pain. lacovides et al. also note that “Women with dysmenorrhea, compared with women without dysmenorrhea, have greater sensitivity to experimental pain both within and outside areas of referred menstrual pain. Importantly, the enhanced pain sensitivity is evident even in phases of the menstrual cycle when women are not experiencing menstrual pain, illustrating that long-term differences in pain perception extend outside of the painful menstruation phase. This enhanced pain sensitivity may increase susceptibility to other chronic pain conditions in later life; dysmenorrhea is a risk factor for fibromyalgia. Further, dysmenorrheic pain has an immediate negative impact on quality of life, for up to a few days every month.”

As noted above, medical practitioners skilled in treating women's health disorders make a clinical distinction between dysmenorrhea, characterized primarily by abdominal pain, such as cramping, and PMDD, which is a depressive (affective) psychological disorder. However, it is also known in the women's health field that dysmenorrhea may also be associated with mental health concerns, including adolescents, young adults, and mature women. For example, it has been shown “that the depression and anxiety levels increased and the psychosocial health subscale scores of quality of life decreased with increasing dysmenorrhea severity” and that for dysmenorrhea management, it is important to enhance awareness among pediatric clinicians and gynecologists regarding the associations between dysmenorrhea and mood problems. See, Sahin et al., “Assessment of anxiety-depression levels and perceptions of quality of life in adolescents with dysmenorrhea,” Reproductive Health 15:13 (2018).

Without being held to a particular mechanism, it is noted that the medical literature suggests that “[s] stress, depressed mood, anxiety, and irritability may be caused by severe pain.” Adib-Rad, et al., “Primary dysmenorrhea associated with psychological distress in medical sciences students in the north of Iran: a cross-sectional study,” Int'l Journal of Fertility and Sterility 16(3): 224-229 (2022). See, also, Pakpour, et al., “Depression, anxiety, stress, and dysmenorrhea: a protocol for a systemic review,” Systemic Reviews 9:65 (2020).

### PMDD and PMS

PMDD is distinguished from premenstrual syndrome (PMS) as described below. According to the DSM.5: “The essential features of premenstrual dysphoric disorder are the expression of mood lability, irritability, dysphoria, and anxiety symptoms that occur repeatedly during the premenstrual phase of the cycle and remit around the onset of menses or shortly thereafter. These symptoms may be accompanied by behavioral and physical symptoms. . . . Typically, symptoms peak around the time of the onset of menses. Although it is not uncommon for symptoms to linger into the first few days of menses, the individual must have a symptom-free period in the follicular phase after the menstrual period begins. While the core symptoms include mood and anxiety symptoms, behavioral and somatic symptoms also commonly occur. However, the presence of physical and/or behavioral symptoms in the absence of mood and/or anxious symptoms is not sufficient for a diagnosis.”

The DSM.5 provides the following diagnostic criteria for PMDD:
“A. In the majority of menstrual cycles, at least five symptoms must be present in the final week before the onset of menses, start to improve within a few days after the onset of menses, and become minimal or absent in the week post menses.
B. One (or more) of the following symptoms must be present:
   1. Marked affective lability (e.g., mood swings; feeling suddenly sad or tearful, or increased sensitivity to rejection).
   2. Marked irritability or anger or increased interpersonal conflicts.
   3. Marked depressed mood, feelings of hopelessness, or self-deprecating thoughts.
   4. Marked anxiety, tension, and/or feelings of being keyed up or on edge.
C. One (or more) of the following symptoms must additionally be present, to reach a total of five symptoms when combined with symptoms from Criterion B above.
   1. Decreased interest in usual activities (e.g., work, school, friends, hobbies).
   2. Subjective difficulty in concentration.
   3. Lethargy, easy fatigability, or marked lack of energy.
   4. Marked change in appetite; overeating; or specific food cravings.
   5. Hypersomnia or insomnia.
   6. A sense of being overwhelmed or out of control.
   7. Physical symptoms such as breast tenderness or swelling, joint or muscle pain, a sensation of “bloating,” or weight gain.
Note: The symptoms in Criteria A-C must have been met for most menstrual cycles that occurred in the preceding year.
D. The symptoms are associated with clinically significant distress or interference with work, school, usual social activities, or relationships with others.
E. The disturbance is not merely an exacerbation of the symptoms of another disorder, such as major depressive disorder, panic disorder, persistent depressive disorder (dysthymia), or a personality disorder (although it may co-occur with any of these disorders).
F. Criterion A should be confirmed by prospective daily ratings during at least two symptomatic cycles. (Note: The diagnosis may be made provisionally prior to this confirmation.)
G. The symptoms are not attributable to the physiological effects of a substance (e.g., a drug of abuse, a medication, other treatment) or another medical condition (e.g., hyperthyroidism).”

The DSM-5 distinguishes PMDD from premenstrual syndrome (PMS) as follows: “Premenstrual syndrome differs from premenstrual dysphoric disorder in that a minimum of five symptoms is not required, and there is no stipulation of affective symptoms for individuals who have premenstrual syndrome. This condition may be more common than premenstrual dysphoric disorder, although the estimated prevalence of premenstrual syndrome varies. While premenstrual syndrome shares the feature of symptom expression during the premenstrual phase of the menstrual cycle, it is generally considered to be less severe than premenstrual dysphoric disorder. The presence of physical or behavioral symptoms in the premenstruum, without the required affective symptoms, likely meets criteria for premenstrual syndrome and not for premenstrual dysphoric disorder.”

Other authors, for example, Cary and Simpson, “Premenstrual disorders and PMDD-a review”, Best Pract. Res. Clin. Endocrinol. Metab., 38, 101858 (2024), using the term premenstrual disorder (PMD) in their article, note a variety of terminology around premenstrual symptoms, commenting that “[I]n lay-speak the terms Premenstrual Tension (PMT) and Premenstrual Syndrome (PMS) have been widely adopted to cover all premenstrual symptoms, often being used interchangeably and without regard for severity and impact,” and “[I]n current medical practice the terms PMS and PMD are both commonly used, with syndrome and disorder used both to de-emphasize the psychological focus of tension but also to convey impact of symptoms on functioning.” They note (citations omitted) that “Worldwide there seems to be consistency in findings that around 90% of women experience at least one premenstrual symptom. When limited to look for PMD with an impact on daily functioning, prevalence falls to between 20-40%. Tightening criteria further to look for PMDD, using the DSM-V criteria that we will outline later in this review, reduces the prevalence even further to around 5%.”

The DSM-5 clearly distinguishes dysmenorrhea from PMDD: “Dysmenorrhea is a syndrome of painful menses, but this is distinct from a syndrome characterized by affective changes. Moreover, symptoms of dysmenorrhea begin with the onset of menses, whereas symptoms of premenstrual dysphoric disorder, by definition, begin before the onset of menses, even if they linger into the first few days of menses.”

### 16α,17α-epoxyestr-4-en-10β-ol-3-one

The compound, 16α,17α-epoxyestr-4-en-10β-ol-3-one, is a neurocircuitry regulator with agonist activity on nasal chemosensory receptors. It belongs to a class of non-hormonal, non-systemic investigational intranasal drug candidates called pherines. Pherines are produced by modification of the chemical structure of natural substances such as androstadienone and estratetraenol secreted by the human skin and are also found in other external secretions (Monti-Bloch, et al., “Effect of putative pheromones on the electrical activity of the human vomeronasal organ and olfactory epithelium,” J. Steroid Biochem. Mol. Biol. 39(4B): 573-82 (1991).

As a class, pherines act as nasal chemosensory receptor agonists that specifically and selectively bind to peripheral receptors in human nasal chemosensory neurons (NCNs) in the nasal passages. Low microgram level doses of pherines administered intranasally in spray form have selective binding affinity to receptors in the NCNs, which rapidly activate olfactory bulb-to-brain neurocircuits without requiring systemic absorption or uptake into the brain to achieve desired therapeutic benefits and differentiated safety. (Monti, et al., “Neural circuits of anxiolytic and antidepressant pherine molecules. CNS Spectr. 27(1): 66-72 (2022). Rapid activation of NCN receptors by 16α,17α-epoxyestr-4-en-10β-ol-3-one stimulates subgroups of microcircuits (glomeruli) in the olfactory bulbs that are connected to the limbic amygdala and the thermoregulatory areas of the hypothalamus.

This understanding of the pharmacological effects of 16α,17α-epoxyestr-4-en-10β-ol-3-one was informed by results from an in vitro receptor binding study that showed no binding to neurotransmitter receptors, re-uptake sites, or steroid receptors. In addition, the receptor binding study suggests that the pharmacological effect of the compound does not bind to GABAA receptors, opioid receptors, or dopamine receptors, thereby limiting its potential for addiction and withdrawal. Moreover, 16α,17αepoxyestr-4 en-10β ol-3 does not bind to estrogen receptors. Given the absence of any substantial systemic uptake and the lack of steroidal hormone receptor binding, it is contemplated that the intranasal administration of 16α,17α epoxyestr-4 en-10β ol-3 one will not be associated with or cause endometrial effects such as endometrial hyperplasia or endometrial cancer as are associated with conventional estrogen therapies.

U.S. Pat. No. 6,057,439 (“U.S. Pat. No. 6,057,439) describes the use of a number of steroidal pherines for the treatment of premenstrual dysphoric disorder and anxiety by administration to the vomeronasal organ of an individual suffering from those symptoms. Among the pherines described in U.S. Pat. 6,057,439 is 16α,17α-Epoxyestr-4-en-10β-ol-3-one, which is also known in the art as PH80, has the following chemical structure:

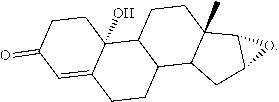 [SMILES: C[C@]12CCC3C(CCC4=CC(=O)CC[C@]43O)C1C[C@H]1O[C@H]12]

U.S. Pat. 6,057,439 also describes the synthesis of 16α,17α-Epoxyestr-4-en-10β-ol-3-one and pharmaceutical compositions containing it for alleviating symptoms of PMDD. U.S. Pat. 6,331,534 describes the same steroids for alleviating pain by vomeronasal administration. U.S. Pat. 8,431,559, “Treatment of hot flashes”, describes a synthesis of 16α,17α-epoxyestr-4-en-10β-ol-3-one, which it refers to as 16α,17α-epoxy-10β-hydroxyestr-4-en-3-one, and a method of using 16α,17α-epoxyestr-4-en-10β-ol-3-one to alleviating hot flashes by administering 16α,17α-epoxyestr-4-en-10β-ol-3-one via intranasal administration. U.S. Pat. No. 11,419,881, “Treatment of migraine”, similarly describes a synthesis of 16α,17α-epoxyestr-4-en-10β-ol-3-one and also refers to the drug as 16α,17α-epoxy-10β-hydroxyestr-4-en-3-one. U.S. Pat. 11,419,881 also describes a method for alleviating migraines by nasally administering 16α,17α-epoxyestr-4-en-10β-ol-3-one.

Preparation of 16α,17α-epoxyestr-4-en-10β-ol-3-one is described in U.S. Pat. No. 6,057,439, and a method for preparing 16α,17α-epoxyestr-4-en-10β-ol-3-one from the readily commercially-available steroid estrone, estra-1,3,5(10)-trien-3-ol-17-one(3-hydroxyestra-1,3,5(10)-trien-17-one), is described in U.S. Pat. Nos. 8,431,559 and 11,419,881.

A person of ordinary skill in the art would not experience any difficulty synthesizing 16α,17α-Epoxyestr-4-en-10β-ol-3-one in view of the disclosures of U.S. Pat. Nos. 6,057,439, 8,431,559, and 11,419,881.

### Treatment of Dysmenorrhea and its Symptoms

Methods of treating dysmenorrhea and the symptoms of dysmenorrhea in subjects by administering 16α,17α-epoxyestr-4-en-10β-ol-3-one to the subjects are contemplated herein. More specifically, in a method of the invention, 16α,17α-epoxyestr-4-en-10β-ol-3-one is administered intranasally to a subject to treat dysmenorrhea. In specific methods of the invention, 16α,17α-epoxyestr-4-en-10β-ol-3-one is administered intranasally to contact chemosensory neurons of the olfactory epithelium of the subject. Accordingly, intranasal administration preferably means administration to the nasal passages in a manner that desirably directs the 16α,17α-epoxyestr-4-en-10β-ol-3-one generally towards the nasal chemosensory neurons, including the receptors in the dorsal nasal cleft area and the vomeronasal organ (“VNO”).

As used in this specification, “comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

The terms “patient,” “individual,” or “subject” are used interchangeably herein, and refer to a mammal, particularly a human female, with pain and functional disruption associated with menstruation. The patient may have a mild, intermediate or severe dysmenorrhea, or at risk of developing dysmenorrhea.

A “therapeutically effective amount” means an amount of 16α,17α-epoxyestr-4-en-10β-ol-3-one that, when administered as a treatment to the nasal chemosensory receptors of an individual suffering from dysmenorrhea, is sufficient to provide a therapeutic benefit for the dysmenorrhea subject, but which amount is insufficient to have a systemic effect on the dysmenorrhea by absorption into the systemic circulation or penetration into the brain.

“Therapeutic benefit” in the context of “treating” or “treatment of” dysmenorrhea includes one or more of:
(1) inhibiting the occurrence of dysmenorrhea, including inhibiting the symptom of severe abdominal cramps or another physical symptom of dysmenorrhea; and/or
(2) relieving the symptoms of dysmenorrhea, including relief of the symptom of severe abdominal cramps or another physical symptom of dysmenorrhea; and/or
(3) preventing the occurrence of the symptoms of dysmenorrhea, including the symptom of severe abdominal cramps or another physical symptom of dysmenorrhea; and/or
(4) inhibiting, relieving, or preventing anxiety, depression, or other affective symptoms that are associated with dysmenorrhea; and/or
(5) improving psychomotor and/or cognitive performance in subjects who have psychological distress, including mental fatigue, associated with dysmenorrhea.

A person of ordinary skill in the art will have no difficulty, considering that skill and this disclosure, determining a therapeutically effective amount of 16α,17α-epoxyestr-4-en-10β-ol-3-one for various intranasal formulations. Preferably, doses are administered well below any level that would cause a clinical or systemic effect other than those effects mediated through the nasal chemosensory neurons.

### Therapeutic Administration

In methods of the invention, 16α,17α-epoxyestr-4-en-10β-ol-3-one is administered intranasally by any suitable route. Routes of administration include, but are not limited to, topical applications (for example, of an intranasal cream or gel), intranasal spray (for example, as described immediately below and in Example 4), intranasal powder spray, mist, aerosol, and the like. Pharmaceutical formulations generally will be formulations designed to administer the drug across mucosal membranes. Suitable formulations for each of these methods of administration may be found, for example, in Gennaro, ed., “Remington: The Science and Practice of Pharmacy”, 20 ed., Lippincott Williams & Wilkins, Philadelphia, Pennsylvania, (2003). Typical preferred formulations will be aqueous solutions for intranasal spray, and will contain 16α,17α-epoxyestr-4-en-10β-ol-3-one and water, typically will also contain one or more other pharmaceutically acceptable excipients to increase the aqueous solubility of the 16α,17α-epoxyestr-4-en-10β-ol-3-one, such as alcohols and glycols (for example, ethanol and propylene glycol) and surfactants (for example, polysorbate 80 (polyoxyethylene (20) sorbitan monooleate)), and may also contain one or more other pharmaceutically acceptable excipients such as buffers, preservatives (antioxidants, chelating agents, etc.), tonicifiers, viscosity enhancers (dextrans, cyclodextrins, polyvinylpyrrolidones, polyethylene glycols, etc.), and the like, such as are well-known for aqueous solutions for intranasal spray.

Suitable delivery devices for these formulations are the metered-dose intranasal spray pumps in common use for intranasal delivery of steroids for allergies and asthma. Such pumps are made by multiple manufacturers, including, for example Aptar (the AptarGroup, Inc.). Liquid volumes should be such that the formulation is efficiently delivered without exceeding the nasal retention volume with an excess either flowing back into the nasal sinuses or dripping from the nose, and a volume of 100 μL has been found convenient, though greater or lesser volumes (50 μL has been satisfactorily tested) will also be satisfactory. For example, in some methods of the invention the delivery device delivers about 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 130 μL, 135 μL, 140 μL, 145 μL, 150 μL, or any other amount from 20 μL to 150 μL of a liquid formulation of containing 16α,17α-epoxyestr-4-en-10β-ol-3-one.

Desirably, the aqueous solution formulations and the delivery devices used to administer them will be chosen to achieve maximum retention of the sprayed formulation within the nasal passages, for example by choosing them such that the mean size of the sprayed droplets will be about 20 microns, 21 microns, 22 microns, 23 microns, 24 microns, 25 microns, 26 microns, 27 microns, 28 microns, 29 microns, 30 microns, 31 microns, 32 microns, 33 microns, 34 microns, 35 microns, 36 microns, 37 microns, 38 microns, 39 microns, 40 microns, 41 microns, 42 microns, 43 microns, 44 microns, 45 microns, 46 microns, 47 microns, 48 microns, 49 microns, 50 microns, 51 microns, 52 microns, 53 microns, 54 microns, 55 microns, 56 microns, 57 microns, 58 microns, 59 microns, or 60 microns. In some methods of the inventions, the administered sprayed droplet size will be from about 30 and 50 microns.

For powder formulations administered in methods of the invention mean size of the administered particles is about 30 microns, 35 microns, 40 microns, 45 microns, 50 microns, 55 microns, 60 microns, 65 microns, 70 microns, 75 microns, 80 microns, 85 microns, 90 microns, 95 microns, 100 microns, 105 microns, 110 microns, 115 microns, 120 microns, or any other size from 30 microns to 120 microns. For example, a particle size from about 50 to 100 microns is generally considered suitable for methods of the invention.

A particularly suitable delivery device for formulations of the invention, especially aqueous solution formulations, containing 16α,17α-epoxyestr-4-en-10β-ol-3-one, is an intranasal drug delivery device that includes: a dispenser assembly including a reservoir; an actuation mechanism including an actuator, the actuator including a base portion, a distal tip portion, and an interior chamber enclosed by a sidewall, wherein: the base portion is attached to the dispenser assembly and is in fluid communication with the reservoir, and the distal tip portion includes a first axial discharge orifice that is in fluid communication with an environment external to the interior chamber; and the sidewall includes a first lateral discharge orifice formed through a thickness of the sidewall that is in fluid communication with the environment external to the interior chamber. See, for example, U.S. patent application Ser. No. 19/172,293, which is incorporated by reference in its entirety.

Another particularly suitable delivery device for formulations of the invention is an intranasal nasal drug delivery device that includes a reservoir that includes an actuator including a lateral sidewall and a distal tip, wherein: the distal tip of the actuator includes or more distal orifices configured to direct a portion of a pharmaceutical substance onto nasal chemosensory receptors associated with the mucosa in the dorsal recess or nasal cleft, and the lateral sidewall of the actuator includes one or more lateral orifices configured to direct a portion of the pharmaceutical substance onto nasal chemosensory receptors associated with the vomeronasal organ.

### Formulations

Exemplary aqueous solution formulations include the two formulations discussed in Example 3 below. A further exemplary formulation is shown in Table 1.

TABLE 1
Exemplary Formulation

| Component | Quantity | | Grade |
|---|---|---|---|
| 16α,17α-epoxyestr-4-en-10β-ol-3-one | 16 | μg/mL | GMP |
| ethanol | 2% | v/v | USP |
| propylene glycol | 3% | v/v | USP |
| polysorbate 80 | 0.5% | w/v | USP |
| benzalkonium chloride 50% | 0.001% | v/v | USP |

| | | |
|---|---|---|
| sterile Water For Injection (WFI) | to 100% | USP |

A person of ordinary skill in the art will have no difficulty, considering that skill and this disclosure, in preparing suitable formulations and delivery systems of 16α,17α-epoxyestr-4-en-10β-ol-3-one for intranasal administration.

A therapeutically effective amount of 16α,17α-epoxyestr-4-en-10β-ol-3-one, when administered in an intranasal spray formulation of the type above, is typically from 0.4 μg to 6.4 μg per administration. Therefore, in certain methods of the invention, the therapeutically effective amount of 16α,17α-epoxyestr-4-en-10β-ol-3-one, when administered in an intranasal spray formulation is about 0.4 μg, 0.5 μg, 0.6 μg, 0.7 μg, 0.8 μg, 0.9 μg. 1 μg. 1.1 μg. 1.2 μg. 1.3 μg, 1.4 μg, 1.5 μg, 1.6 μg, 1.7 μg, 1.8 μg, 1.9 μg, 2 μg, 2.1 μg, 2.2 μg, 2.3 μg, 2.4 μg, 2.5 μg, 2.6 μg, 2.7 μg, 2.8 μg, 2.9 μg, 3 μg, 3.1 μg, 3.2 μg, 3.3 μg, 3.4 μg, 3.5 μg, 3.6 μg, 3.7 μg, 3.8 μg, 3.9 μg, 4 μg, 4.1 μg, 4.2 μg, 4.3 μg, 4.4 μg, 4.5 μg, 4.6 μg, 4.7 μg, 4.8 μg, 4.9 μg, 5 μg, 5.1 μg, 5.2 μg, 5.3 μg, 5.4 μg, 5.5 μg, 5.6 μg, 5.7 μg, 5.8 μg, 5.9 μg, 6 μg, 6.1 μg, 6.2 μg, 6.3 μg, and 6.4 μg. In some preferable methods of the invention, from 0.8 μg to 3.2 μg of 16α,17α-epoxyestr-4-en-10β-ol-3-one is delivered per administration, for example 1.6 μg per administration.

With the foregoing descriptions in mind, for a 16 mg/L (16 ppm) concentration of 16α,17α-epoxyestr-4-en-10β-ol-3-one in the formulation, each administration would therefore be 100 μl of the formulation, conveniently a single administration of 100 μL to one nostril (or one-half that per nostril, if the compound were to be administered to both nostrils; though the use of the 100 μL volume is convenient because it allows for doubling the administration simply by administering one spray to each nostril).

Initial pharmacological response to the intranasal administration of 16α,17α-epoxyestr-4-en-10β-ol-3-one takes place very rapidly in symptomatic individuals or subjects, typically within seconds to a minute after administration. A more complete pharmacological response occurs slightly more slowly; but an effect on dysmenorrhea (or on one or more symptoms of dysmenorrhea) is expected within 5 to 15 minutes.

Because of the rapid onset of effect and safety of intranasally administered 16α,17α-epoxyestr-4-en-10β-ol-3-one, it is expected that the compound may be administered pro re nata (that is “PRN” or “as needed,” for example, when an individual senses the onset of an episode of dysmenorrhea, to pre-emptively inhibit or to mitigate the dysmenorrhea symptoms, or when the individual senses a frank symptom of an episode of dysmenorrhea (such as uterine cramping or other symptoms described in the subsection, above, entitled “Dysmenorrhea”) to relieve and palliate the symptoms of that episode.

It is expected that a formulation of 16α,17α-epoxyestr-4-en-10β-ol-3-one may be administered more than once, such as over a period of a few days, if it is first administered to prevent or minimize the dysmenorrhea or a symptom thereof, by scheduled administration throughout the day, such as 1, 2, 3, 4, 5, or 6 times/day (for a total daily dose of 0.4 to 38.4 μg per day), for example from 2 to 6 times/day, or 3 to 5 times/day, such as 4 times/day. This scheduled administration may be on a uniform schedule, for example at 8 a.m., noon, 4 p.m., and 8 p.m. (for 4 times/day administration), or on a non-uniform schedule where the administration is chosen to maximize the administration at the time when the expected occurrence of dysmenorrhea symptoms is most frequent or most severe, either in the dysmenorrhea symptomatic population or in the individual being treated. Thus, for example, administration might be at 9 a.m., 3 p.m., 5 p.m., and 8 p.m. (again for 4 times/day) to maximize the administration at the time when the expected occurrence of symptoms is most frequent.

The present inventor surprisingly found that 16α,17α-epoxyestr-4-en-10α-ol-3-one also reduces the severity of one or more symptoms of dysmenorrhea when it is administered in a preventative or prophylactic mode prior to the onset of dysmenorrhea in an affected subject. In this mode of the invention, a prophylactically effective amount of 16α,17α-epoxyestr-4-en-10β-ol-3-one may be administered 1, 2, 3, 4, or 5 days before the expected start of a symptom or symptoms of dysmenorrhea, especially for subjects who consistently experience dysmenorrhea.

It is contemplated that the administration of 16α,17α-epoxyestr-4-en-10β-ol-3-one may continue during the period of time that a subject experiences one or more symptoms of dysmenorrhea. Without being held to a definitive mechanism of action for such prophylactic use, it is believed that the plasticity of certain hypothalamic neural circuits allows a certain amount of neurological resetting by administration of the 16α,17α epoxyestr-4 en-10β ol-3 one.

Of course, even if scheduled administration is being used, it is possible to administer the compound on an as-needed basis if symptoms are still experienced. However, because of its rapid action, it is contemplated that there will not be a need to use it chronically: treatment can be interrupted when the individual no longer senses a dysmenorrhea symptom or the risk of a dysmenorrhea symptom and then, resumed when it is next needed. However, if there are also symptoms of PMDD or PMS in an individual with dysmenorrhea, that individual may start treatment with intranasally administered 16α,17α-epoxyestr-4-en-10β-ol-3-one at the onset of the PMDD/PMS symptoms and continue treatment until the end of the dysmenorrhea symptoms.

Also, because of the rapid onset of effect of intranasally administered 16α,17α-epoxyestr-4-en-10β-ol-3-one, it is expected that the compound may be administered as an adjunct to conventional (e.g. oral or injectable) therapy for dysmenorrhea. For example, it may be used as “rescue” medication in individuals who may still suffer from an episode of a symptom of dysmenorrhea even while on conventional therapy for dysmenorrhea, to relieve and palliate the symptoms of that episode as needed.

Thus, intranasally administered 16α,17α-epoxyestr-4-en-10β-ol-3-one may be used acutely or intermittently, and alone and in combination with conventional therapy for dysmenorrhea, in the treatment of dysmenorrhea.

### Adjunctive Treatment

It is contemplated that the compositions of 16α,17α-epoxyestr-4-en-10β-ol-3-one as described in the specification, may be administered in an adjunctive manner, either concurrently or as otherwise scheduled, with other pharmaceutical products, for example, COX inhibitors or antidepressants, hormonal contraceptives of progestin if NSAIDs are not effector or appropriate, and Vitamin B1 and magnesium supplements that may be prescribed to treat dysmenorrhea or its symptoms.

### Combination Therapy

It is further contemplated that the compositions of 16α,17α-epoxyestr-4-en-10β-ol-3-one, as described in the specification, may be co-administered (or co-formulated) with other pherine compounds that are effective in treating the affective symptoms of anxiety or depression associated with dysmenorrhea. These include fasedienol (4,16-androstadien-3β-ol), currently in clinical development for the treatment of social anxiety disorder (see U.S. Pat. No. 8,309,539), and itruvone (pregn-4-en-20-yn-3-one), currently in clinical development for the treatment of major depressive disorder (see U.S. Pat. No. 10,322,138). Similarly, it is contemplated that the compositions of 16α,17α-epoxyestr-4-en-10β-ol-3-one, as described in the specification, may be co-administered (or co-formulated) with other pherine compounds that would be effective in improving psychomotor and/or cognitive performance in subjects who have mental fatigue associated with dysmenorrhea. These include PH15 (1,3,5(10),16-estratetraen-3-yl acetate), which has demonstrated effectiveness in clinical trials (see U.S. application Ser. No. 18/732,200).

The invention further contemplates a package or kit that combines a ready-for-nasal administration device together with 16α,17α-epoxyestr-4-en-10β-ol-3-one and appropriate packaging and instructions for its use according to the methods described in this specification.

### EXAMPLES

### Example 1: Electrophysiological Studies With 16α,17α-epoxyestr-4-en-10β-ol-3-one

16α,17α-Epoxyestr-4-en-10β-ol-3-one induced inward currents in isolated human nasal chemosensory neurons and electrotonic depolarization of the nasal septal chemosensory mucosa: the first event in chemotransduction in peripheral receptors. The amplitude of this response increased depending on the concentration of the compound, with a maximal response at 10⁻⁷ to 10⁻⁶ M. In vitro, there was no agonist or antagonist activity on estrogen, androgen, progestin and glucocorticoid receptors, nor was there any affinity for receptors of indoleamine, monoamines, ion channels, receptors, peptides, opioid, glutamate neurotransmitter, steroid hormones, or glucocorticoid receptors, suggesting that the compound exerts its effects through specific receptors that are different from the other described binding sites.

### Example 2: Preclinical Studies With 16α,17α-epoxyestr-4-en-10β-ol-3-one

Acute and multidose (28 day) toxicity studies in rats, mice, rabbits and dogs with single (up to 100 μg/rat, 400 μg/rabbit, 600 μg/dog) and repeated (up to 50 μg/rat/day, 300 μg/dog/day) intranasal doses and single and repeated intravenous doses of 16α,17α-epoxyestr-4-en-10β-ol-3-one (up to 2.5 mg/kg/day in rats and rabbits) (up to 2.5 mg/kg in rats, mice, rabbits) demonstrated that the compound was well tolerated in all species tested, with no deaths or adverse clinical signs or effects on laboratory or pathology parameters observed.

Genotoxicity tests revealed no evidence of mutagenic or clastogenic potential of the compound when examined in the Ames reverse mutation assay and the in vivo bone marrow micronucleus test. Reproductive toxicity studies in pregnant rabbits revealed no adverse effects on maternal or litter parameters attributable to the compound at intravenous doses up to 2.5 mg/kg/day administered during the period of organogenesis. Preclinical pharmacokinetic studies with the compound demonstrated very low systemic exposure when the compound was administered by repeated or singe escalating intranasal doses up to 100 μg/rat, 400 μg/rabbit, 600 μg/dog. When given to rats, rabbits, or dogs in single repeated intravenous doses up to 2.5 mg/kg, plasma concentrations of the compound generally were dose-proportional and decreased rapidly.

### Example 3: Preliminary Human Studies With 16α,17α-epoxyestr-4-en-10β-ol-3-one

Preliminary findings showed that intranasal administration of microgram quantities of 16α,17α-epoxyestr-4-en-10β-ol-3-one to human volunteers of both sexes induced concentration dependent activation of the electrogram recorded from the surface of the nasal chemosensory mucosa. The half effective dose of this effect was 0.087 μg. The electrogram induced during intranasal administration of 1.6 μg of 16α,17α-epoxyestr-4-en-10β-ol-3-one was rapidly followed (latency 5-10 seconds) by modulation of reflex activity integrated in the limbic system, hypothalamus, and cerebral cortex. There was significantly increased amplitude of the depth of the inspiratory cycle of respiration and decreased respiration rate within physiologic range, increased amplitude of the skin conductance measured as electrodermal activity or dermo-limbic reflex from the palmar surface of the 4^{th} and 5^{th} finger, decreased skeletal muscle tone (relaxation) measured from the electromyogram recorded transdermally from the chin muscles, and decreased body core temperature recorded from the external ear canal. There was no significant change in cardiac rate.

The effects of 16α,17α-epoxyestr-4-en-10β-ol-3-one on physiologic parameters are explained as follows:
(a) decreased respiration rate and increased depth of respiration are the result of decreased resistance of the airways induced by bronchiolar dilation. 16α,17α-Epoxyestr-4-en-10β-ol-3-one decreased parasympathetic system tone resulting in increased sympathetic activity and stimulation of β₂ adrenergic receptors in the bronchiole smooth muscle that induced bronchodilation;
(b) increased skin conductance amplitude is the result of increased skin secretions, which are produced by preponderance of the sympathetic nervous system tone (or decreased parasympathetic tone);
(c) decreased body temperature is explained by increased activity of the posterior (adrenergic) hypothalamus and decreased parasympathetic tone (anterior hypothalamus). This effect was also shown in menopausal women as decreased number, severity and bother of hot flashes after intranasal administration of 16α,17α-epoxyestr-4-en-10β-ol-3-one; and
(d) decreased muscle tone or relaxation was induced through the efferent limbic-hypothalamic neural connections to the cortical areas.

### Example 4: Human Clinical Study With 16α,17α-epoxyestr-4-en-10β-ol-3-one to Evaluate Changes in Physiological Parameters

In a group of 14 women of reproductive age, intranasal administration of a nasal spray containing 0.5 μg of 16α,17α-epoxyestr-4-en-10β-ol-3-one per administration (administration by an Aptar intranasal spray pump of 50 μL of an aqueous solution of 10 μg/mL 16α,17α-epoxyestr-4-en-10β-ol-3-one with 2% propylene glycol and 2% ethanol), compared to the vehicle alone, induced statistically significant decreases in respiratory frequency, skeletal muscle tone, galvanic skin reflex, and core body temperature; and a not statistically significant increase in cardiac frequency.

### Example 5: Human Clinical Study With 16α,17α-epoxyestr-4-en-10β-ol-3-one to Evaluate Changes in Tone of the Sympathoadrenergic System

In another study also in women of reproductive age, intranasal delivery of a nasal spray containing 1.6 μg of 16α,17α-epoxyestr-4-en-10β-ol-3-one (one administration per nostril by an Aptar intranasal spray pump of 50 μL of an aqueous solution of 16 μg/mL 16α,17α-epoxyestr-4-en-10β-ol-3-one with 2% propylene glycol and 2% ethanol) induced a rapid (0.5 to 4 minute latency) decrease in core body temperature of 1±0.23° C. that persisted for 9±2.5 minutes. It also changed the tone of the sympathoadrenergic system (as assessed by measuring physiologic sinus arrhythmia) within 5 minutes of administration, and the effect persisted for 15 to 20 minutes.

### Example 6: Human Clinical Studies With 16α,17α-epoxyestr-4-en-10β-ol-3-one to Evaluate Symptoms of PMDD Using the Penn Daily Symptom Report

In a randomized, double-blind, placebo-controlled test in women diagnosed with PMDD, where the women began treatment on the first day of “bother” of their PMDD (Day 1) and continued treatment for six days or until the first day of menses (so that Day 6 is the presumptive start of menses), the women were asked to self-rate their symptoms using the Penn Daily Symptom Report (17 questions, rating mood, physical symptoms, and amount of impairment of social activities, family relationships, and/or work for the previous 24 hour period, with each symptom being rated on a five point scale from 0 (“not present at all”) to 4 (“very severe”). In this test, intranasal administration of an aerosol containing 0.9 μg of 16α,17α-epoxyestr-4-en-10β-ol-3-one and 3 mg dextrose as diluent (one administration in one nostril by an Aptar non-pressurized spring-activated disposable single dose nasal applicator, using air as a carrier) induced significant improvements in hypothalamic (fatigue, anger, craving for foods), limbic system (mood swings, depression, lack of interest) and cortical (headache, tension) behavioral symptoms.

The foregoing improvements appeared within 30 minutes after single dose intranasal administration of the formulation and persisted for approximately 5 hours. Because of the excellent safety profile of 16α,17α-epoxyestr-4-en-10β-ol-3-one and lack of systemic exposure demonstrated in a pharmacokinetic bioavailability study, the medication was repeated up to 4 times daily. No serious adverse events were reported and the side effects were minimal and similar to administration of placebo. Therefore, intranasal administration of 16α,17α-epoxyestr-4-en-10β-ol-3-one starting at the onset of behavioral symptoms of PMDD activates neural circuits in the limbic-hypothalamic areas of the brain that improves behavioral functioning (mood swings, anger, tension, stress), which are related to perception of pain.

### Example 7: Improvement of Cramping Symptoms in the PMDD Clinical Study of Example 6

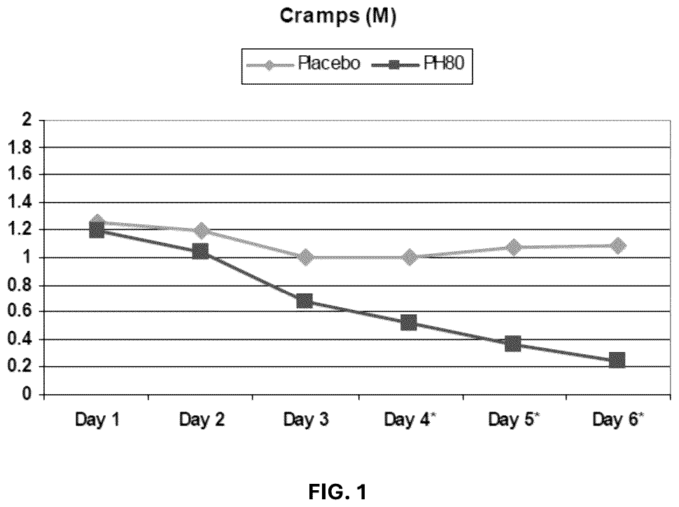

As seen in FIG. 1, at Day 1, the 16α,17α-epoxyestr-4-en-10β-ol-3-one group (29 women) and the placebo group (23 women) had mean cramping scores of 1.19 and 1.24, respectively. By Day 4, a significant (p<0.05, one-tail test) difference in the mean scores had appeared; and by Day 6, the mean cramping scores were 0.24 and 1.10; with only 17% of the 16α,17α-epoxyestr-4-en-10β-ol-3-one group reporting cramping symptoms, while 84% of the placebo group still reported cramping symptoms. Thus, on days 5 and 6, when cramping caused by dysmenorrhea may be expected to begin/occur, women treated with 16α,17α-epoxyestr-4-en-10β-ol-3-one displayed significantly less cramping than women treated with placebo; and it is possible that there is both an acute benefit and a cumulative benefit given the steady decrease in cramping score. In the figure, 16α,17α-epoxyestr-4-en-10β-ol-3-one is denoted by “PH80”.

### Example 8: Improvement of Headache Symptoms in the PMDD Clinical Study of Example 6

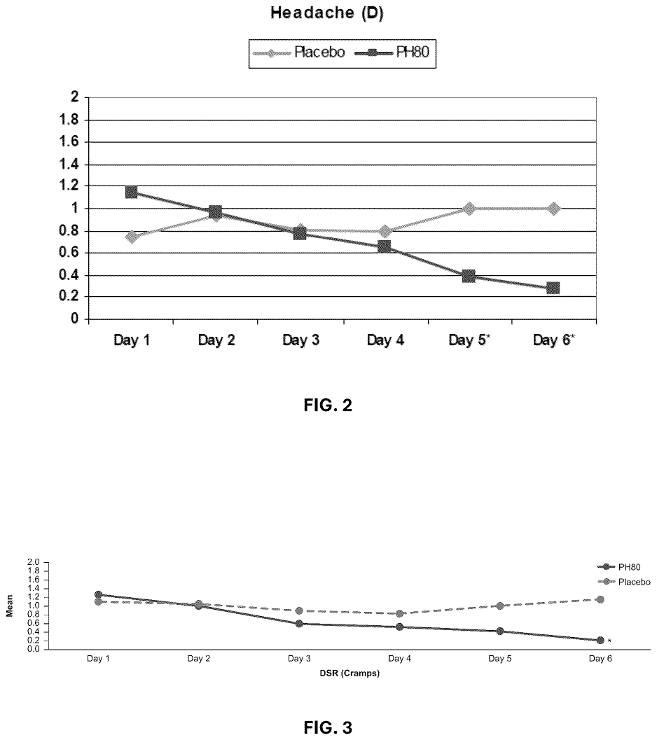

As seen in FIG. 2, at Day 1, the 16α,17α-epoxyestr-4-en-10β-ol-3-one group and the placebo group had mean headache scores of 1.14 and 0.74 respectively. The scores crossed at Day 2; by Day 5, a significant (p<0.05, one-tail test) difference in the mean scores had appeared; and by Day 6, the mean headache scores were 0.28 and 1.00. Thus, on days 5 and 6, when headache caused by dysmenorrhea may be expected to begin/occur, women treated with 16α,17α-epoxyestr-4-en-10β-ol-3-one displayed significantly less headache than women treated with placebo. In the figure, 16α,17α-epoxyestr-4-en-10β-ol-3-one is denoted by “PH80”.

The foregoing physiological and symptomatic effects, as shown by the clinical data of Examples 6 to 8, including, especially, a decrease in cramping, headache, and fatigue (being symptoms of dysmenorrhea noted by Mckenna and Fogleman), as well as decreased skeletal muscle tension, decreased galvanic skin reflex frequency of events, and decrease in tension, depression, irritability/anger, anhedonia, and food cravings, are predictive of the efficacy of the nasal administration of 16α,17α-epoxyestr-4-en-10β-ol-3-one in the treatment of dysmenorrhea.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

### Example 9: Improvement of Cramps in Pain Symptom Cluster Analysis From a Daily Symptom Report in a Phase 2a Study

Females aged 18 to 40 years with ≥1-year history of DSM-IV-defined PMDD were enrolled in a clinical study of the effects of 16α,17α-epoxyestr-4-en-10β-ol-3-one (PH80) administration on pain symptoms, assessing changes in headache, aches, and cramps using the daily symptom report (DSR) pain cluster score. During a 3- to 5-week screening period, patients who presented to the clinic for bothersome PMDD symptoms (defined as “affected functioning”) were recruited. The Premenstrual Tension Syndrome scale was used for screening. After study eligibility criteria and PMDD symptom presence were confirmed at Visit 1, patients received single-blind placebo; those with no acute improvement were eligible for randomization study. Randomized patients presented for Visit 2 when PMDD symptoms were bothersome during the second cycle after entry. PH80 (0.9 μg) or placebo was administered intranasally; PMDD symptoms were evaluated 30 and 60 minutes later. Patients were then supplied with their assigned double-blind treatment for intranasal self-administration up to 4 times a day (PH80 0.9 μg/dose or matching placebo) for up to 6 consecutive days before the estimated menses onset in that cycle. On the first day of menses in the second cycle, a follow-up visit (Visit 3) occurred. 82 females were screened; 61 met eligibility criteria and were randomized to receive PH80 (n=33) or placebo (n=28). Seven patients failed to return for Visit 2 and/or pick up study medication, and 2 were lost to follow-up. A total of 49 patients provided at least 1 post-treatment assessment and were included in the pain efficacy analysis.

Pain efficacy analysis for cramping was rated daily by the patient on a 5-point scale ranging from 0 (no symptoms at all) up to 4 (very severe). The overall pain cluster score and individual symptom ratings were compared post-hoc using two-sided T-tests, without adjustments for multiplicity or within-patient variance; means for each group were plotted for each reported day, and no imputation was made for missing data. Results are summarized in Table 2 and depicted in FIG. 3. At Day 6, PH80 (n=19) demonstrated statistically significant improvement over placebo (n=13) on the cramps line item (0.21 for PH80 vs 1.15 for placebo; P=0.01) (FIG. 3).

TABLE 2
Summary of Pain Efficacy Analysis for Cramps

| | Day 1 | Day 2 | Day 3 | Day 4 | Day 5 | Day 6 |
|---|---|---|---|---|---|---|
| Placebo mean | 1.10 | 1.05 | 0.89 | 0.82 | 1.00 | 1.15 |
| Placebo SD | 0.97 | 0.91 | 1.08 | 0.88 | 1.25 | 1.28 |
| PH80 Mean | 1.26 | 1.00 | 0.59 | 0.52 | 0.42 | 0.21 |
| PH80 SD | 1.32 | 1.04 | 0.84 | 0.80 | 0.81 | 0.71 |
| P-value | 0.65 | 0.86 | 0.31 | 0.24 | 0.08 | 0.01 |

### Example 10: Improvement of Feelings of Depression Associated With Dysmenorrhea

Females aged 18 to 40 are administered a therapeutic amount of itruvone in combination with a therapeutic amount of 16α,17α-epoxyestr-4-en-10α-ol-3-one. Improvement is recorded through use, for example, of the Hamilton Rating Scale for Depression (HRSD-17) as described in U.S. Patent No. U.S. Pat. No. 10,322,138, and through use of other evaluation tests known to persons skilled in the art.

### Example 11: Improvement in Psychomotor Performance in Subjects Having Dysmenorrhea

Females aged 18 to 40 are administered a therapeutic amount of PH15 in combination with a therapeutic amount of 16α,17α-epoxyestr-4-en-10α-ol-3-one. Improvement is recorded through use, for example, of a computerized Reaction Time and Time Estimation Test (RTTET) as described in U.S. application Ser. No. 18/732,200 and through use of other evaluation tests known to persons skilled in the art.

1. A method of treating dysmenorrhea in an individual, comprising the intranasal administration of an amount of 16α,17α-epoxyestr-4-en-10β-ol-3-one that is therapeutically effective to inhibit, prevent or relieve the occurrence of one or more symptoms of dysmenorrhea in that individual.

2. The method of claim 1, wherein the intranasal administration is preferentially administered to the olfactory epithelium in the nasal cavity.

3. The method of claim 1, wherein the dysmenorrhea is primary dysmenorrhea.

4. The method of claim 1, wherein the dysmenorrhea is secondary dysmenorrhea.

5. The method of claim 1, wherein the 16α,17α-epoxyestr-4-en-10β-ol-3-one is administered at the onset of a symptom of dysmenorrhea.

6. The method of claim 5, wherein the 16α,17α-epoxyestr-4-en-10β-ol-3-one is administered 1 to 6 times per day, 2 to 5 times per day, or 3 or 4 times per day.

7. The method of claim 5, wherein the 16α,17α-epoxyestr-4-en-10β-ol-3-one is administered as scheduled or as needed.

8. The method of claim 5, wherein the 16α,17α-epoxyestr-4-en-10β-ol-3-one is administered partly as scheduled and partly as needed.

9. The method of claim 1, wherein the frequency of administration or the administered dose of the 16α,17α-epoxyestr-4-en-10β-ol-3-one is increased when a symptom of dysmenorrhea is perceived by the individual being treated as relatively more severe.

10. The method of claim 1, wherein the frequency of administration or the administered dose of the 16α,17α-epoxyestr-4-en-10β-ol-3-one is increased when a symptom of dysmenorrhea is expected to be relatively more severe based on the treated individual's historical course of symptomatology.

11. The method of claim 1, wherein the frequency of administration or the administered dose of the 16α,17α-epoxyestr-4-en-10β-ol-3-one is increased when a symptom of dysmenorrhea is expected to be relatively more severe based on the course of symptomatology in the general dysmenorrhea symptomatic population.

12. The method of claim 1, wherein the 16α,17α-epoxyestr-4-en-10β-ol-3-one is administered in a nasal spray.

13. The method of claim 12, wherein the nasal spray comprises an aqueous solution of 16α, 17α-epoxyestr-4-en-10β-ol-3-one.

14. The method of claim 13, wherein the 16α,17α-epoxyestr-4-en-10β-ol-3-one content of the nasal spray is about 0.8 to 6.4 μg per administration.

15. The method of claim 14, wherein the 16α,17α-epoxyestr-4-en-10β-ol-3-one content of the nasal spray is about 1.6 to 3.2 μg per administration.

16. The method of claim 15, wherein the 16α,17α-epoxyestr-4-en-10β-ol-3-one content of the nasal spray is about 1.6 μg per administration.

17. A method of prophylactically treating an individual before the onset of dysmenorrhea, comprising the intranasal administration of an amount of 16α,17α-epoxyestr-4-en-10β-ol-3-one that is prophylactically effective to inhibit, prevent or relieve the occurrence of one or more symptoms of dysmenorrhea in that individual.

18. The method of claim 17, wherein the 16α,17α-epoxyestr-4-en-10β-ol-3-one is administered prophylactically 1, 2, 3, 4, or 5 days before the expected onset of a symptom of dysmenorrhea.

19. The method of claim 18, wherein the 16α,17α-epoxyestr-4-en-10β-ol-3-one content of the nasal spray is about 0.8 to 6.4 μg per administration.

20. The method of claim 1, further comprising combining the administration of the therapeutic amount of 16α,17α-epoxyestr-4-en-10β-ol-3-one with a therapeutic amount of fasedienol or itruvone, either separately or together with the 16α,17α-epoxyestr-4-en-10β-ol-3-one, to further treat symptoms of anxiety, depression, or psychological distress associated with dysmenorrhea in the individual.

21. The method of claim 1, further comprising combining the administration of the therapeutic amount of 16α,17α-epoxyestr-4-en-10β-ol-3-one with a therapeutic amount of PH15 (1,3,5(10),16-estratetraen-3-yl acetate), either separately or together with the 16α,17α-epoxyestr-4-en-10β-ol-3-one, to improve cognitive performance or psychomotor performance, wherein the individual experiences mental fatigue associated with dysmenorrhea.

22. A nasal spray device for the administration of 16α,17α-epoxyestr-4-en-10β-ol-3-one packaged together with a label for the symptomatic or prophylactic treatment of the symptoms of dysmenorrhea.
